(19) United States
(12) Patent Application Publication
(10) Pub. No.: US 2005/0191251 A1
(43) Pub. Date: Sep. 1, 2005

(54) Anhydrous bleaching paste comprising at least one ceramide-type compound and method using it

(75) Inventors: Sylvain Kravtchenko, Asnieres (FR); Frederic Legrand, Courbevoie (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/017,732

(22) Filed: Dec. 22, 2004

Related U.S. Application Data
(60) Provisional application No. 60/534,370, filed on Jan. 6, 2004.

(30) Foreign Application Priority Data
Dec. 29, 2003 (FR) 0351216

(51) Int. Cl. A61K 7/135; A61K 33/40

(52) U.S. Cl. 424/62; 424/616

(57) ABSTRACT

Paste for bleaching human keratinous fibres, and more particularly the hair, containing at least one organic inert liquid, at least one peroxygenated salt and at least one alkaline agent, wherein it comprises at least one ceramide-type compound. Method for bleaching human keratinous fibres using it, and an appropriate device.

### REFERENCE TO PRIOR APPLICATIONS

This application claims priority to U.S. provisional application No. 60/534,370 filed Jan. 6, 2004, and to French patent application 0351216 filed Dec. 29, 2003, both incorporated herein by reference.

### FIELD OF THE INVENTION

One subject of the present invention is an anhydrous paste for bleaching human keratinous fibres, comprising at least one ceramide-type compound. It further relates to a method using the said composition and an appropriate multicompartment device.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Human keratinous fibres, and more particularly the hair, are bleached by oxidation of the “melanin” pigment, resulting in the solubilization and partial or complete removal of this pigment.

To bleach the hair, a bleaching composition, in powdered form or more advantageously in anhydrous paste form, containing at least one peroxygenated reagent, is used which is combined immediately before use with an aqueous hydrogen peroxide composition.

The peroxygenated salts and hydrogen peroxide being relatively stable in an acid medium, it is often necessary to activate them at basic pH in order to obtain sufficient formation of oxygen. It is therefore customary to add to the bleaching composition alkaline compounds such as urea, alkali or alkaline-earth metal silicates and phosphates or ammonia precursors.

The problem encountered with the use of this type of composition consists in the fact that in the long run, degradation of the keratinous fibre is observed. This results in particular in a reduction in the cosmetic properties of the fibre, a loss of softness for example, and in a reduction in the mechanical properties of the said fibre. Indeed, the fibre is embrittled and a degree of breakage of the fibre during disentanglement, for example, is observed.

It is proposed to use bleaching compositions in powdered form, comprising persalts and a ceramide-type compound. While such compositions constitute an improvement compared with ceramide-free pulverulent compositions, the results obtained are however not completely satisfactory.

### OBJECTS OF THE INVENTION

One object of the present invention is therefore to provide compositions which make it possible to intensely lighten the hair while limiting their degradation and while offering enhanced safety and improved performance in terms of lightening and comfort.

### DETAILED DESCRIPTION OF THE PREFERRED EMBODIMENTS

These aims and others are achieved by the present invention whose first subject is an anhydrous paste for bleaching human keratinous fibres, and more particularly the hair, comprising at least one organic inert liquid, at least one peroxygenated salt and at least one alkaline agent, wherein it comprises at least one ceramide-type compound.

Another subject of the invention is a method for bleaching human keratinous fibres, and in particular the hair, comprising:
mixing, immediately before use, an anhydrous paste according to the invention, with an aqueous hydrogen peroxide composition,
applying the mixture obtained to the area of the fibres to be bleached,
leaving in for a sufficient period to obtain the desired bleaching,
removing the bleaching mixture by rinsing with water followed by optional washing with a shampoo, and then optionally drying.

The invention relates furthermore to a multicompartment device for bleaching human keratinous fibres, and more particularly the hair, wherein it comprises at least two compartments of which one comprises or and/is configured to comprise an anhydrous paste according to the invention, and the other comprises and/or is configured to comprise an aqueous hydrogen peroxide composition.

However, other characteristics and advantages of the present invention will emerge more clearly on reading the description and example which follow.

As indicated above, the anhydrous paste which is a subject of the invention comprises at least one ceramide-type (ceramide) compound.

As used herein the term “anhydrous paste” denotes a paste whose water content is less than 1%, and preferably less than 0.5% by weight relative to the total weight of the paste.

More particularly, the ceramide-type (ceramide) compounds of the invention refer to and includes compounds chosen from ceramides and/or glycoceramides and/or pseudoceramides.

According to an advantageous embodiment of the present invention, the ceramide-type compound is chosen from the compounds corresponding to the following formula (A): 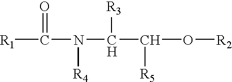
in which:
R₁ denotes:
   either a saturated or unsaturated, linear or branched C₁-C₅₀, preferably C₅-C₅₀, hydrocarbon radical, it being possible for this radical to be substituted with one or more hydroxyl groups optionally esterified with an acid R₇COOH, R₇ being an optionally mono- or polyhydroxylated, linear or branched, saturated or unsaturated C₁-C₃₅ hydrocarbon radical, it being possible for the hydroxyl(s) of the radical R₇ to be esterified with an optionally mono- or polyhydroxylated, linear or branched, saturated or unsaturated C₁-C₃₅ fatty acid,
   or a radical R″—(NR—CO)—R′, R denotes a hydrogen atom or a mono- or polyhydroxylated, preferably monohydroxylated, C₁-C₂₀ hydrocarbon radical, R′ and R″ are hydrocarbon radicals of which the sum of the carbon atoms is between 9 and 30, R′ being a divalent radical,
   or a radical R₈—O—CO—(CH₂)ₚ, R₈ denoting a C₁-C₂₀ hydrocarbon radical, p being an integer varying from 1 to 12;
R₂ is chosen from a hydrogen atom, a saccharide-type radical, in particular a (glycosyl)ₙ, (galactosyl)ₘ and sulphogalactosyl radical, a sulphate or phosphate residue, a phosphorylethylamine radical and a phosphorylethylammonium radical, in which n is an integer varying from 1 to 4 and m is an integer varying from 1 to 8;
R₃ denotes a hydrogen atom or a hydroxylated or nonhydroxylated, saturated or unsaturated, C₁-C₃₃ hydrocarbon radical, it being possible for the hydroxyl(s) to be esterified with an inorganic acid or an acid R₇COOH, R₇ having the same meanings as above, and it being possible for the hydroxyl(s) to be etherified with a (glycosyl)ₙ, (galactosyl)ₘ, sulphogalactosyl, phosphorylethylamine or phosphorylethylammonium radical, it being also possible for R₃ to be substituted with one or more C₁-C₁₄ alkyl radicals;
R⁴ denotes a hydrogen atom, a methyl or ethyl radical, an optionally hydroxylated, linear or branched, saturated or unsaturated C₃-C₅₀ hydrocarbon radical or a radical —CH₂—CHOH—CH₂—O—R₆ in which R₆ denotes a C₁₀-C₂₆ hydrocarbon radical or a radical R₈—O—CO—(CH₂)ₚ, R₈ denotes a C₁-C₂₀ hydrocarbon radical, p being an integer varying from 1 to 12,

R₅ denotes a hydrogen atom or an optionally mono- or polyhydroxylated, linear or branched, saturated or unsaturated C₁-C₃₀ hydrocarbon radical, it being possible for the hydroxyl(s) to be etherified with a (glycosyl)ₙ, (galactosyl)ₘ, sulphogalactosyl, phosphorylethylamine or phosphorylethylammonium radical,

with the proviso that when R₃ and R₅ denote hydrogen or when R₃ denotes hydrogen and R₅ denotes methyl, then R₄ does not denote a hydrogen atom, or a methyl or ethyl radical.

Among the compounds of formula (A) above, there may be mentioned most particularly the ceramides and/or glycoceramides described by DOWNING in Journal of Lipid Research, Vol. 35, page 2060, 1994 or those described in French patent application FR-2 673 179, and whose teachings are included herein by way of reference.

Among the preferred ceramide-type compounds, there may be mentioned those for which, in formula (A), R₁ denotes an optionally hydroxylated, saturated or unsaturated alkyl radical derived from C₁₄-C₂₂ fatty acids; R₂ denotes a hydrogen atom; and R₃ denotes an optionally hydroxylated, saturated, linear C₁₁-C₁₇, preferably C₁₃-C₁₅ radical.

Such compounds are for example chosen from, alone or as a mixture:
N-linoleoyldihydrosphingosine,
N-oleoyldihydrosphingosine,
N-palmitoyldihydrosphingosine,
N-stearoyldihydrosphingosine,
N-behenoyldihydrosphingosine,
N-2-hydroxypalmitoyldihydrosphingosine,
N-stearoylphytosphingosine,
N-palmitamidohexadecanediol.

It is also possible to use specific mixtures such as for example mixtures of ceramide(s) 2 and of ceramide(s) 5 according to the DOWNING classification.

It is also possible to use the compounds of formula (A) for which R₁ denotes a saturated or unsaturated alkyl radical derived from fatty acids; R₂ denotes a galactosyl or sulphogalactosyl radical; and R₃ denotes a saturated or unsaturated C₁₂-C₂₂ hydrocarbon radical and preferably a group —CH═CH—(CH₂)₁₂—CH₃.

Ceramide-type compounds are for example described in patent applications DE 4424530, DE 4424533, DE 4402929, DE 4420736, WO 95/23807, WO 94/07844, EP 646572, WO 95/16665, FR 2673179, EP 227994 and WO 94/07844, WO 94/24097, WO 94/10131 to which reference may be made.

By way of example, there may be mentioned the product consisting of a mixture of glycoceramides, sold under the trade name Glycocer® by the company Waitaki International Biosciences.

It is also possible to use the compounds described in patent applications EP 227994, EP 647617, EP 736522 and WO 94/07844.

Such compounds are for example Questamide H®, also called bis(N-hydroxyethyl-N-cetyl)malonamide and sold by the company Quest and cetylic acid N-(2-hydroxyethyl)-N-(3-cetyloxy-2-hydroxypropyl)amide.

It is also possible to use N-docosanoyl-N-methyl-D-glucamine as described in international application WO 94/24097.

According to an advantageous embodiment of the invention, the content of ceramide-type compound represents from 0.001 to 5% by weight, more particularly from 0.01 to 3% by weight and preferably from 0.05 to 1% by weight relative to the weight of the anhydrous paste.

In addition to the ceramide-type compound, the anhydrous bleaching paste comprises at least one inert organic liquid.

More particularly, the organic inert liquid is chosen from polydecenes of formula C₁₀ₙH_{[(20n)+2]} in which n varies from 3 to 9 and preferably from 3 to 7, fatty acid esters of fatty alcohols, C₁₂-C₂₄ fatty acid diesters of sugars, cyclic ethers or cyclic esters, silicone oils, mineral oils or vegetable oils.

The compounds of formula C₁₀ₙH_{[(20n)+2]}, with n varying from 3 to 9, correspond to the name “polydecene” in the CTFA Dictionary 7th edition 1997 by the Cosmetic, Toiletry and Fragrance Association, USA, and to the same I.N.C.I. name in the USA and in Europe.

They are products of hydrogenation of poly-1-decenes.

Among these compounds, those for which, in the formula, n varies from 3 to 7 are more particularly chosen according to the invention.

There may also be mentioned, by way of example, and preferably, the product sold under the name Silkflo® 366 NF Polydecene by the company Amoco Chemical, those sold under the name Nexbase® 2002 FG, 2004 FG, 2006 FG and 2008 FG by the company Fortum.

As regards the fatty acid esters of alcohols, there may be mentioned in particular:
esters of linear or branched saturated C₃-C₆ low monoalcohols, with monofunctional C₁₂-C₂₄ fatty acids (it being possible for the latter to be saturated or unsaturated, linear or branched and chosen in particular from oleates, laurates, palmitates, myristates, behenates, cocoates, stearates, linoleates, linolenates, caprates, arachidonates, or mixtures thereof, such as in particular oleopalmitates, oleostearates, palmitostearates, and the like). Among the said esters, the use of isopropyl palmitate, isopropyl myristate and octyldodecyl stearate is more particularly preferred,
esters of linear or branched C₃-C₈ monoalcohols with bifunctional C₈-C₂₄ fatty acids (it being possible for the latter to be saturated or unsaturated, linear or branched), such as for example diisopropyl ester of sebacic acid (diisopropyl sebacate),
esters of linear or branched C₃-C₈ monoalcohols, with bifunctional C₂-C₈ fatty acids (it being possible for the latter to be saturated or unsaturated, linear or branched), such as for example dioctyl adipate and dicaprylyl maleate,
the ester of a trifunctional acid such as triethyl citrate.

The inert organic liquid may also be chosen from the C₁₂-C₂₄ fatty acid esters and diesters of sugars.

The expression “sugar” is understood to mean compounds which possess several alcohol functional groups, with or without an aldehyde or ketone functional group, and which contain at least 4 carbon atoms. These sugars may be monosaccharides, oligosaccharides or polysaccharides.

As sugars which can be used according to the invention, there may be mentioned for example sucrose, glucose, galactose, ribose, fucose, maltose, fructose, mannose, arabinose, xylose, lactose, and their derivatives, in particular their alkylated derivatives, such as the methylated derivatives such as methylglucose.

The fatty acid esters of sugars which can be used according to the invention may be chosen in particular from the group comprising the esters or mixtures of esters of sugars described above and of saturated or unsaturated, linear or branched C₁₂-C₂₄ fatty acids.

The esters may be chosen from mono-, di-, tri- and tetraesters, polyesters and mixtures thereof.

The said esters may be chosen for example from oleates, laurates, palmitates, myristates, behenates, cocoates, stearates, linoleates, linolenates, caprates, arachidonates, or mixtures thereof, such as in particular the mixed esters oleopalmitates, oleostearates, palmitostearates and the like.

More particularly, the use of mono- and diesters, and in particular mono- or dioleates, stearates, behenates, oleopalmitates, linoleates, linolenates, oleostearates, of sucrose, of glucose or of methylglucose is preferred.

There may also be mentioned, by way of example, and preferably, the product sold under the name Glucate® DO by the company Amerchol, which is a methylglucose dioleate.

There may also be mentioned, by way of examples of fatty acid esters or mixtures of fatty acid esters of sugar:
the products sold under the names F160, F140, F 110, F90, F70, SL40 by the company Crodesta, designating respectively the sucrose palmitostearates formed of 73% of monoester and 27% of di- and triester, of 61% of monoester and 39% of di-, tri- and tetraester, of 52% of monoester and 48% of di-, tri- and tetraester, of 45% of monoester and 55% of di-, tri- and tetraester, of 39% of monoester and 61% of di-, tri- and tetraester, and sucrose monolaurate;
the products sold under the name Ryoto Sugar Esters for example designated by the reference B370 and corresponding to sucrose behenate formed of 20% of monoester and 80% of di-triester-polyester;
the sucrose mono-di-palmito-stearate marketed by the company Goldschmidt under the name Tegosoft® PSE.

As regards the cyclic ethers and cyclic esters, γ-butyrolactone, dimethyl isosorbide or diisopropyl isosorbide are especially suitable.

Silicone oils may also be used as inert organic liquid.

More particularly, the suitable silicone oils are liquid and nonvolatile silicone fluids having a viscosity of less than or equal to 10 000 mPa.s at 25° C., the viscosity of silicones being measured according to the ASTM 445 standard, Annex C.

Silicone oils are defined in greater detail in the manual by Walter NOLL “Chemistry and Technology of Silicones” (1968)—Academic Press.

Among the silicone oils which can be used according to the invention, there may be mentioned in particular for example the silicone oils sold under the names DC-200 fluid—5 mPa.s, DC-200 fluid—20 mPa.s, DC-200 fluid—350 mPa.s, DC-200 fluid—1000 mPa.s, DC-200 fluid—10 000 mPa.s by the company Dow Corning.

Mineral oils may also be used as organic inert liquid, such as for example paraffin oil.

Vegetable oils may also be suitable, in particular avocado oil, olive oil or liquid jojoba wax.

Preferably, the organic inert liquid is chosen from the group consisting of the polydecenes of formula C₁₀ₙH_{[(20n)+2]} in which n varies from 3 to 9 and preferably from 3 to 7, and fatty acid esters of fatty alcohols.

Moreover, in accordance with one embodiment of the invention, the content of organic inert liquid varies from 5 to 60% by weight, preferably from 10 to 50% by weight relative to the weight of the anhydrous paste.

As indicated above, the anhydrous paste according to the invention comprises at least one peroxygenated salt.

Advantageously, the latter is chosen from persulphates, perborates and percarbonates of ammonium or alkali metals, such as potassium, sodium; magnesium peroxide, alone or as a mixture.

Preferably, the anhydrous paste comprises at least one persulphate as peroxygenated salt, and still more preferably, at least one sodium and potassium persulphate.

In accordance with one variant of the invention, the peroxygenated salt content varies from 10 to 70% by weight, preferably from 20 to 60% by weight relative to the weight of the anhydrous paste.

The anhydrous paste according to the invention further comprises at least one alkaline agent. Preferably, this agent is chosen from urea, and alkali or alkaline-earth metal silicates, phosphates and bicarbonates, and in particular alkali metal metasilicates, or ammonia precursors such as ammonium salts, for example chlorides, sulphates, phosphates or nitrates.

Usually, the content of alkaline agent varies from 0.01 to 40% by weight, preferably from 0.1 to 30% by weight relative to the weight of the anhydrous paste.

The anhydrous paste according to the invention may even comprise other additives customarily used in the field.

The bleaching paste may thus comprise, for example, water-soluble thickening polymers, fillers such as clays or amorphous silica, binders such as vinylpyrrolidone, lubricants such as polyol stearates or alkali or alkaline-earth metal stearates, and agents controlling the release of oxygen such as magnesium carbonate or oxide, colouring agents or mattifying agents such as titanium oxides or anionic, nonionic, cationic or amphoteric surfactants, vitamins.

By way of illustration, the content of additive(s) represents 0.01 to 40% by weight, preferably from 0.1 to 30% by weight relative to the total weight of the composition.

The anhydrous bleaching paste which has just been described may be obtained by dispersing, under mechanical action, all the pulverulent compounds in the organic inert liquid in which the other liquid compounds of the composition have been dispersed or mixed beforehand.

It is also possible to prepare the paste by extrusion, by introducing the liquid and solid phases of the composition into the extruder and then by mixing them at a temperature of less than 25° C. with the aid of a twin-screw co-rotating system composed of transporting and blending components.

The anhydrous bleaching paste which has just been described is advantageously used for the preparation of a ready-to-use composition which results from freshly mixing the said paste with an aqueous hydrogen peroxide composition.

The anhydrous paste is thus mixed with about 0.5 to 10 equivalents by weight of an aqueous hydrogen peroxide composition which may be a solution, a direct or invert emulsion, or a gel, having a hydrogen peroxide concentration by weight ranging from 2 to 12%.

This mixture should preferably be prepared immediately before applying the product to the hair.

The aqueous hydrogen peroxide composition preferably has a pH of less than 7; the acid pH ensuring the stability of the hydrogen peroxide in this composition.

It may be obtained by means of acidifying agents such as for example hydrochloric acid, acetic acid, ethydronic acid, phosphoric acid, lactic acid or boric acid, and it may be conventionally adjusted by adding either basifying agents, such as for example aqueous ammonia, monoethanolamine, diethanolamine, triethanolamine, isopropanolamine, 1,3-diaminopropane, an alkali metal or ammonium (bi)carbonate, an organic carbonate such as guanidine carbonate, or an alkali metal hydroxide, it being of course possible for all these compounds to be taken alone or as a mixture.

The aqueous hydrogen peroxide composition may also contain preservatives, colorants, perfumes, antifoaming agents, hydrogen peroxide stabilizing agents such as in particular sodium pyrophosphate, sodium stannate and sodium salicylate as well as sequestering agents such as for example ethylenediaminetetraacetic acid (EDTA) or pentasodium pentetate (CTFA name).

Of course, persons skilled in the art will be careful to choose the possible additional compound(s) mentioned above so that the advantageous properties intrinsically attached to the anhydrous bleaching paste or to the ready-to-use bleaching composition according to the invention are not, or not substantially impaired by the addition(s) envisaged.

The pH of the ready-to-use bleaching composition is generally between the values 4 and 12. It is preferably between 7 and 11.5, and still more preferably between 8 and 11.

The bleaching method according to the invention consists in mixing, immediately before use, an anhydrous paste as described, with an aqueous hydrogen peroxide composition. Next, the ready-to-use bleaching composition thus obtained is applied to the dry or wet area of the human keratinous fibres to be bleached, and is left in for a sufficient leave-in time in order to obtain the desired bleaching.

In general, this period varies from about 1 to 60 minutes, and preferably from about 1 to 30 minutes.

Usually, the temperature at which the composition is applied is of the order of 15 to 80° C. and preferably of 15 to 40° C.

Once the desired bleaching has been obtained, the bleaching mixture is removed by rinsing the fibres with water, preferably followed by at least one washing with a shampoo, and then optionally drying.

According to another aspect, the subject of the invention is also a multicompartment device for bleaching human keratinous fibres, and more particularly the hair, comprising at least two compartments of which one contains an anhydrous paste according to the invention, and the other an aqueous hydrogen peroxide composition.

A concrete but nonlimiting example will now be presented.

### EXAMPLE

The following composition was prepared:

| | Composition |
|---|---|
| | (g) |
| Steareth-100/PEG-136/HMDI copolymer | 2.0 |
| Isopropyl myristate | 21.6 |
| Xanthan gum | 1.4 |
| Ultramarines | 0.5 |
| Magnesium oxide | 2.0 |
| Sodium silicate | 15.0 |
| Titanium dioxide | 1.0 |
| N-oleyldihydrosphingosine | 0.01 |
| Magnesium stearate | 2.0 |
| EDTA | 0.2 |
| Sodium lauryl sulphate | 4.0 |
| Silica | 1.0 |
| Potassium persulphate | 39.08 |
| Sodium persulphate | 6.0 |
| Beeswax | 1.2 |
| Mineral oil | 1.0 |
| Sodium salt of carboxymethylated starch | 2.0 |

The composition was mixed with the oxidant 30 volumes.

The oxidant had the following composition:

| | |
|---|---|
| Sodium stannate | 0.04 |
| Sodium pentetate | 0.06 |
| Cetearyl alcohol | 8.00 |
| Ceteareth-33 | 2.00 |
| Phosphoric acid | qs pH 3 |
| Hydrogen peroxide | 9.00 |
| Sodium pyrophosphate | 0.03 |
| Acrylate/beheneth-25 methacrylate copolymer | 0.40 |
| (Aculyn 28 ® marketed by Rhom & Haas) | |
| Water | 80.47 |

The mixture was easy to obtain and to apply. It was spread over locks of chestnut brown natural hair of 2.7 g.

The bath ratio was 10 and the leave-in time was 35 minutes, after which the locks were rinsed and shampooed.

It is evident that the hair had an excellent degree of bleaching.

The above written description of the invention provides a manner and process of making and using it such that any person skilled in this art is enabled to make and use the same, this enablement being provided in particular for the subject matter of the appended claims, which make up a part of the original description and including an anhydrous paste for bleaching human keratinous fibres, and more particularly the hair, comprising at least one organic inert liquid, at least one peroxygenated salt and at least one alkaline agent, wherein it comprises at least one ceramide-type compound. Preferred embodiments of the invention similarly fully described and enabled include a method for bleaching human keratinous fibres, and in particular the hair, comprising:
mixing, immediately before use, an anhydrous paste defined according to any one of Claims 1 to 11, with an aqueous hydrogen peroxide composition,
applying the mixture obtained to the area of the fibres to be bleached,
leaving in for a sufficient period to obtain the desired bleaching,
removing the bleaching mixture by rinsing with water followed by optional washing with a shampoo, and then optionally drying.

Similarly enabled is a multicompartment device for bleaching human keratinous fibres, and more particularly the hair, wherein it comprises at least two compartments of which one comprises an anhydrous paste according to the invention, and the other an aqueous hydrogen peroxide composition.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. An anhydrous paste comprising at least one organic inert liquid, at least one peroxygenated salt, at least one alkaline agent, and at least one ceramide compound.

2. An anhydrous paste according to claim 1, wherein the ceramide compound is selected from the group consisting of ceramides, glycoceramides, pseudoceramides and mixtures thereof.

3. An anhydrous paste according to claim 1, wherein the ceramide compound is selected from the group consisting of compounds corresponding to the following formula (A): 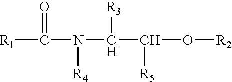
in which:
R₁ denotes:
either a saturated or unsaturated, linear or branched C₁-C₅₀, preferably C₅-C₅₀, hydrocarbon radical, it being possible for this radical to be substituted with one or more hydroxyl groups optionally esterified with an acid R₇COOH, R₇ being an optionally mono- or polyhydroxylated, linear or branched, saturated or unsaturated C₁-C₃₅ hydrocarbon radical, it being possible for the hydroxyl(s) of the radical R₇ to be esterified with an optionally mono- or polyhydroxylated, linear or branched, saturated or unsaturated C₁-C₃₅ fatty acid,
or a radical R″—(NR—CO)—R′, R denotes a hydrogen atom or a mono- or polyhydroxylated, preferably monohydroxylated, C₁-C₂₀ hydrocarbon radical, R′ and R″ are hydrocarbon radicals of which the sum of the carbon atoms is between 9 and 30, R′ being a divalent radical,
or a radical R₈—O—CO—(CH₂)ₚ, R₈ denoting a C₁-C₂₀ hydrocarbon radical, p being an integer varying from 1 to 12;
R₂ is chosen from a hydrogen atom, a saccharide radical, a sulphate or phosphate residue, a phosphorylethylamine radical and a phosphorylethylammonium radical, in which n is an integer varying from 1 to 4 and m is an integer varying from 1 to 8;
R₃ denotes a hydrogen atom or a hydroxylated or nonhydroxylated, saturated or unsaturated, C₁-C₃₃ hydrocarbon radical, it being possible for the hydroxyl(s) to be esterified with an inorganic acid or an acid R₇COOH, R₇ having the same meanings as above, and it being possible for the hydroxyl(s) to be etherified with a (glycosyl)ₙ, (galactosyl)ₘ, sulphogalactosyl, phosphorylethylamine or phosphorylethylammonium radical, it being also possible for R₃ to be substituted with one or more C₁-C₁₄ alkyl radicals;
R₄ denotes a hydrogen atom, a methyl or ethyl radical, an optionally. hydroxylated, linear or branched, saturated or unsaturated C₃-C₅₀ hydrocarbon radical or a radical —CH₂—CHOH—CH₂—O—R₆ in which R₆ denotes a C₁₀-C₂₆ hydrocarbon radical or a radical R₈—O—CO—(CH₂)ₚ, R₈ denotes a C₁-C₂₀ hydrocarbon radical, p being an integer varying from 1 to 12,
R₅ denotes a hydrogen atom or an optionally mono- or polyhydroxylated, linear or branched, saturated or unsaturated C₁-C₃₀ hydrocarbon radical, it being possible for the hydroxyl(s) to be etherified with a (glycosyl)ₙ, (galactosyl)ₘ, sulphogalactosyl, phosphorylethylamine or phosphorylethylammonium radical,
with the proviso that when R₃ and R₅ denote hydrogen or when R₃ denotes hydrogen and R₅ denotes methyl, then R₄ does not denote a hydrogen atom, or a methyl or ethyl radical,
and mixtures thereof.

4. An anhydrous paste according to claim 1, wherein the content of ceramide compound represents from 0.001 to 5% by weight relative to the weight of the anhydrous paste.

5. An anhydrous paste according to claim 1, wherein the organic inert liquid is selected from the group consisting of polydecenes of formula C₁₀ₙH_{[(20n)+2]} in which n varies from 3 to 9, fatty acid esters of fatty alcohols, C₁₂-C₂₄ fatty acid diesters of sugars, cyclic ethers or cyclic esters, silicone oils, mineral oils, vegetable oils, and mixtures thereof.

6. An anhydrous paste according to claim 5, wherein the organic inert liquid is selected from the group consisting of polydecenes of formula C₁₀ₙH_{[(20n)+2]} in which n varies from 3 to 9, fatty acid esters of fatty alcohols, and mixtures thereof.

7. An anhydrous paste according to claim 1, wherein the content of organic inert liquid is from 5 to 60% by weight relative to the weight of the anhydrous paste.

8. An anhydrous paste according to claim 1, wherein the peroxygenated salt is selected from the group consisting of persulphates, perborates and percarbonates of ammonium or alkali metals, magnesium peroxide, and mixtures thereof.

9. An anhydrous paste according to claim 8, wherein the peroxygenated salt content is from 10 to 70% by weight relative to the weight of the anhydrous paste.

10. An anhydrous paste according to claim 1, wherein the alkaline agent is selected from the group consisting of urea, silicates, phosphates and bicarbonates of alkali or alkaline-earth metals, ammonia precursors, and mixtures thereof.

11. An anhydrous paste according to the claim 10, wherein the content of alkaline agent is from 0.01 to 40% by weight relative to the weight of the anhydrous paste.

12. A method for bleaching human keratinous fibres, comprising:
mixing, before use, an anhydrous paste defined according to claim 1 with an aqueous hydrogen peroxide composition to provide a bleaching mixture,
applying the bleaching mixture obtained to an area of the fibres to be bleached,
leaving in the bleaching mixture for a sufficient period to obtain bleaching,
removing the bleaching mixture by rinsing with water optionally followed by washing with a shampoo, and then optionally drying.

13. A method according to claim 12, wherein said anhydrous paste is an an anhydrous paste defined according to claim 2.

14. A method according to claim 12, wherein said anhydrous paste is an an anhydrous paste defined according to claim 3.

15. A method according to claim 12, wherein said anhydrous paste is an an anhydrous paste defined according to claim 4.

16. A method according to claim 12, wherein said anhydrous paste is an an anhydrous paste defined according to claim 5.

17. A method according to claim 12, wherein said anhydrous paste is an an anhydrous paste defined according to claim 6.

18. A method according to claim 12, wherein said anhydrous paste is an an anhydrous paste defined according to claim 7.

19. A method according to claim 12, wherein said anhydrous paste is an an anhydrous paste defined according to claim 8.

20. A multicompartment device for bleaching human keratinous fibres, wherein it comprises at least two compartments of which one comprises an anhydrous paste according to claim 1, and the other an aqueous hydrogen peroxide composition.
